(19) United States
(12) Patent Application Publication
(10) Pub. No.: US 2011/0106034 A1
(43) Pub. Date: May 5, 2011

(54) SYSTEM AND METHOD FOR DISPOSING OF FEMININE HYGIENE WASTE

(76) Inventor: Khristal A. Kramer, Lorain, OH (US)

(21) Appl. No.: 12/611,244

(22) Filed: Nov. 3, 2009

(51) Int. Cl. A61F 13/551; A61L 15/46

(52) U.S. Cl. 604/359; 604/385.13

(57) ABSTRACT

The present invention relates to a system for the disposal feminine hygiene waste. The feminine hygiene waste disposal system specifically includes a sleeve with sealable ends and containing a bag designed for the deposition of used or discarded feminine hygiene waste.

### TECHNICAL FIELD OF THE INVENTION

This invention relates to a system and method for disposing feminine hygiene waste and more specifically to a disposable feminine hygiene waste device that is portable and incorporates a sleeve with a disposable hag that receives the feminine hygiene waste and is then enclosed in the sleeve for easy disposal.

### BACKGROUND OF THE INVENTION

Feminine hygiene products like tampons or sanitary napkins are necessarily worn during this period of the menstrual cycle to absorb and block a woman's menstrual flow. In general, such items must be replaced frequently. Moreover, the need to remove and replace a sanitary hygiene product often arises abruptly or unexpectedly, when the hygiene product's absorbent material become saturated or nearly saturated.

The discreet disposal of feminine hygiene products poses a tricky dilemma for women experiencing menstruation. The act of removing and disposing of these sanitary hygiene products calls for a level of privacy that is difficult to find, particularly outside of one's home. The disposal of these sanitary hygiene products in litter bins is often unsightly, odoriferous and unhygienic. On the other hand, disposing of such hygiene products into a toilet or other septic receptacle is prone to cause blockage and overflow of the plumbing components.

Several methods and apparatuses have been developed to cope with this issue. At their most basic these consist of bundling the hygiene product in toilet or tissue paper or placing it in the product wrapper, then disposing of it into a trash receptacle. This method does not seal off the sanitary hygiene product completely, and fluids or odors may still escape easily. Alternatively, some women use such methods to retain the used hygiene product on their person, for example in their purse, until a satisfactory disposal receptacle can be found. This method also suffers from the lack of an effective seal around the feminine hygiene product and could result in the hygiene product or its contained fluids coming into contact with other items in such purse.

Some public restrooms feature separate waste containers designated for the disposal of feminine hygiene products. These containers are often covered with a lid and, in theory, limit the exposure of disposed feminine hygiene products to others using the designated receptacle. These too however, fail to effectuate a complete seal around the hygiene products. Moreover such receptacles can be especially burdensome or unpleasant for janitorial staff or others tasked with cleaning them or replacing the garbage bags lining their interior.

Other public restrooms provide bags or baggies for the disposal of feminine hygiene products. As with other technologies and methods, this solution also fails to engender a tight seal around the product, allowing odor or fluid to escape.

In response to this persistent issue, several inventions have attempted to alleviate the difficulties associated with the disposal of feminine hygiene products. For example, U.S. Pat. No. 7,422,106 to Kendra discloses a “feminine hygiene product storage and waste disposal system . . . adapted for use in storage of feminine hygiene products prior to use and for the disposal of feminine hygiene products after use.”

### SUMMARY OF THE INVENTION

The present invention relates to a system and method for disposing of feminine hygiene waste. The system and method for disposing of feminine hygiene waste entails the use of a sleeve enclosing a hag to contain and dispose of feminine hygiene waste.

The prior art in this field has failed to redress some of the most pressing problems in feminine hygiene waste disposal. Specifically, under the existing prior art, a discarded used feminine hygiene product may be unsightly or odorous. Additionally a feminine hygiene product disposed under prior art technologies may leak onto or come into unwanted contact with other refuse or the disposal bin or liner itself. Moreover, the prior art has generally failed to produce a method or device for the disposal of feminine hygiene waste that achieves the necessary portability or compactness. An aspect of the present invention is to provide an effective, discreet and clean system for the disposal of feminine hygiene waste.

According to the present invention, the disposable feminine hygiene waste disposal device comprises a sleeve having an upper open end and a lower open end, an upper means extending outward from the upper open end for closing the upper open end of the sleeve, lower means extending outward from the lower open end for closing the lower open end of the sleeve, and a disposal bag within the sleeve adapted to contain feminine hygiene waste.

The disposable feminine hygiene waste disposal device of the present invention may also include a device with a sleeve having a waterproof interior surface. Additionally, the inner surface may contain a deodorizing element.

In addition, according to the present invention, the disposable feminine hygiene waste disposal device encompasses a device wherein the upper means comprises a pair of upper flaps that may each fold to engage each other. The disposable feminine hygiene waste disposal device can also describe a device wherein the lower means comprises a pair of lower flaps that may fold to engage each other. Such lower flaps can be folded when sealed so that the sleeve can be flattened.

Further according to the present invention, the disposal bag of the device may be attached to an interior surface of the sleeve by means of an adhesive strip. Additionally the disposal bag may be constructed of waterproof material and feature a means for sealing the open end of the bag. The bag may also have a tab projecting outward from upper open end of the sleeve.

The present invention also includes a method for disposing of feminine hygiene waste, comprising the steps of providing a sleeve having an upper open end with upper flaps extending outward from the upper open end for closing the upper open end of the sleeve and a lower open end with lower flaps extending outward from the lower open end for closing the lower open end of the sleeve, providing a disposal bag within the sleeve, closing the lower open end of the sleeve by folding the lower flaps to engage each other, disposing feminine hygiene waste within the disposable bag, closing the upper open end of the sleeve by folding the upper flaps to engage each other whereby the disposable bag is contained entirely within the sleeve.

Further according to the present invention, the method includes a process wherein the sleeve has a waterproof interior surface.

Additionally, according to the present invention, the method can incorporate the sleeve with an inner surface containing a deodorizing element.

Still further according to the present invention, under the proposed method, the lower flaps can also be folded when sealed so that the sleeve can be flattened.

Yet further according to the present invention, under the proposed method the disposal bag may adhere to an inner surface of the sleeve or be constructed of waterproof material.

Moreover according to the present invention, under the method, the disposal bag features a means for sealing the open end of the bag. The proposed method may also encompass a method wherein a tab on the disposal bag projects outward from upper open end of the sleeve.

### BRIEF DESCRIPTION OF THE DRAWINGS

The operation, and advantages of the present invention will become apparent upon consideration of the description herein taken in conjunction with the accompanying figures. The figures are intended to be illustrative, not limiting. Certain elements in some of the figures may be omitted, or illustrated not-to-scale, for illustrative clarity.

In the drawings accompanying the description that follows, both reference numerals and legends (labels, text descriptions) may be used to identify elements. If legends are provided, they are intended merely as an aid to the reader, and should not in any way be interpreted as limiting.

Although the invention is generally described in the context of these preferred embodiments, it should be understood that the figures are not intended to limit the spirit and scope of the invention to these particular embodiments.

The figures submitted include the following:

FIG. 1 is a front facing view of the outer facing side of the sleeve of the sanitary hygiene disposal system prior to assembly.

FIG. 2 is a front facing view of the inner facing side of the sleeve of the sanitary hygiene disposal system prior to assembly.

FIG. 2A is a cut-away, perspective view of the bag.

FIG. 3 is a perspective view of the hygiene disposal system assembled and in a flattened condition, with one end opened and the opposite end closed.

FIG. 4 is a front facing view of the feminine hygiene disposal system assembled and in a flattened condition with one end opened.

FIG. 5 is a perspective view of the hygiene disposal system assembled, with both ends shut.

### DETAILED DESCRIPTION OF THE PREFERRED EMBODIMENTS

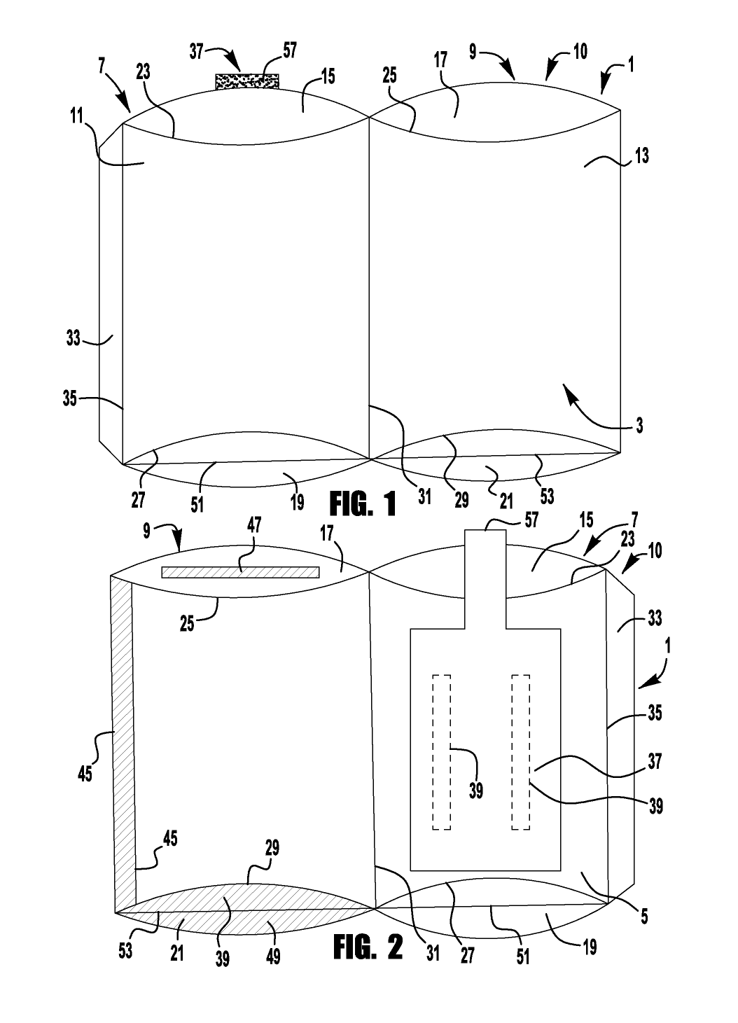

Referring to the drawings, FIG. 1 illustrates the sleeve 1 of an embodiment of a feminine hygiene disposal system 10. In this view the sleeve 1 is disassembled and laid flat, to show its exterior surface 3. The sleeve 1 could be constructed from a variety of materials, selected for water resistance, durability, portability and/or strength. Additionally, the exterior surface 3 could come in a variety of textures, colors or patterns to enhance its cosmetic appeal.

The exterior surface 3 consists of a front side 7 and a similarly shaped reverse side 9. When constructed the front side 7 and reverse side 9 join to form the sleeve 1, as shown in FIG. 3.

In the embodiment shown in FIG. 1, sides 7 and 9 are comprised of first and second lateral faces 11 and 13, respectively, with top flaps 15 and 17, respectively, and bottom flaps 19 and 21, respectively, at the top and bottom ends of the first and second lateral faces 11 and 13, respectively. Fold lines 23, 25, 27, and 29 separate the first and second lateral faces 11 and 13 from the top and bottom flaps 15, 17, 19, and 21, respectively, and serve to guide the user in the assembly of the sleeve 1, as shown in FIG. 3.

One side of both the first and second lateral faces 11 and 13 are joined along a fold line 31. The first lateral face 11 is additionally joined to a side attachment tab 33 along the side opposite the second lateral face 13. These two surfaces, the front lateral face 11 and the side attachment tab 33, are joined along a fold line 35. Additional fold lines 51 and 53 bisect the bottom flaps 19 and 21.

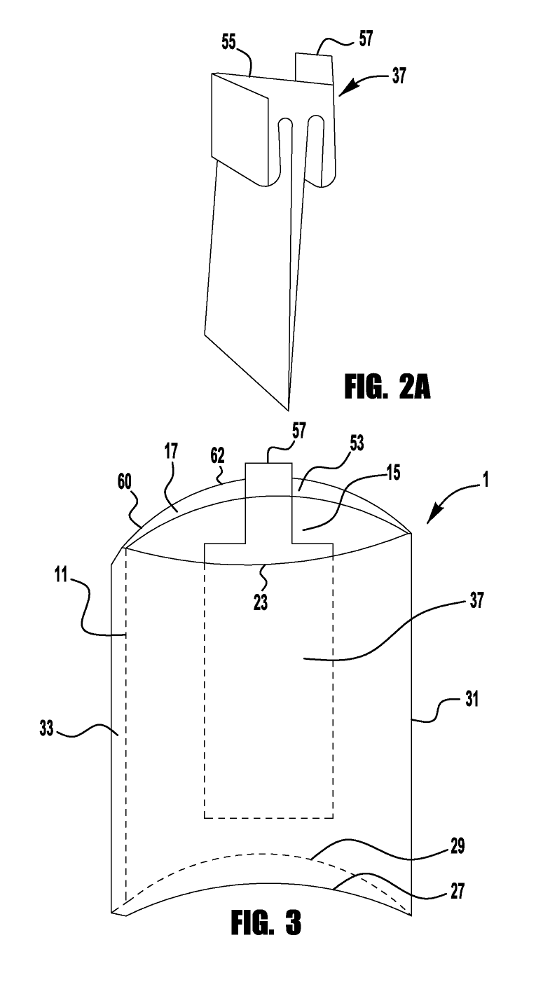

FIG. 2 illustrates the sleeve 1 of the feminine hygiene disposal system 10, disassembled and laid flat, to show the interior surface 5 of sleeve 1. Note that FIG. 2 is FIG. 1 turned around so that the flap 33 is on the right side instead of the left side of the drawing. The interior surface 5 may itself be waterproof, or may incorporate a waterproof element such as disposable bag 37. Additionally the inner surface 5 may contain a deodorizing element (not shown), such as for example a packet containing a deodorant material within the disposable bag 37 or secured by any means, such as an adhesive, to the interior surface 5 of the sleeve 1.

Disposal bag 37, as shown in FIGS. 2 and 2A, is disposed within the assembled sleeve 1. The sleeve 37 can either be loosely contained within the sleeve 1 or mounted therein as shown in FIG. 2.

In FIG. 2, disposal bag 37 can be attached, for example, by means of two adhesive strips 39 to the interior surface 5 of the front side 7. Additionally strips of adhesive 45, 47, and 49 can be disposed on the outer edge of the reverse side 9 of the lateral face 13 and on the top and bottom flaps 17 and 21. The adhesive strips may be composed of material that retains its adhesive properties even when moist or wet. One or more of the adhesive strips may be covered with liner, such that the adhesive surface can be activated, at the election of the user, by peeling away the liner. Alternatively the adhesive may be pressure sensitive, only adhering effectively to surfaces when pressed tightly against it.

FIG. 2A illustrates the disposal bag 37. Bag 37 is constructed of material selected for waterproof, odor-proof and durable qualities. Additionally the bag 37 itself may be lined with deodorant or absorbent substances. The disposal bag 37, on its open end 55, may feature a means to seal or reseal the bag tightly, such as a Ziploc seal, adhesive or tie ends. In addition, the bag can include a tab 57 attached to the open end and adapted to project from the open end of sleeve as discussed hereinafter.

A method to assemble the feminine hygiene disposal system 10 as embodied in the above description and corresponding figures can be practiced as follows. The sleeve 1 is folded medially at fold line 31 bringing the interior surface 5 into near contact with itself. The side attachment tab 33 is folded along fold line 35 and tucked underneath the reverse lateral face 13, and is pressed against the adhesive strip 45 so that the front and reverse sides 7 and 9, respectively, form a sleeve 1 encompassing a hollow compartment 60 with openings 62 and 64 on the upper and lower ends. The front bottom flap 19 is then folded inward as to cover the bottom opening, then the reverse bottom flap 21 is folded inward over the front bottom flap. The adhesive strip 49 affixes the outside surface of the front bottom flap 19 to the inside surface of the reverse bottom flap 21.

At this point the disposal system is assembled and the sleeve 1 is formed with a hollow compartment with a single opening 53 at the upper end, as shown in FIG. 3. This compartment contains the disposal bag 37. A tab 57 on the disposal bag 37 may protrude outward from the opening 53 to facilitate the opening, closing or other manipulation of the disposal bag.

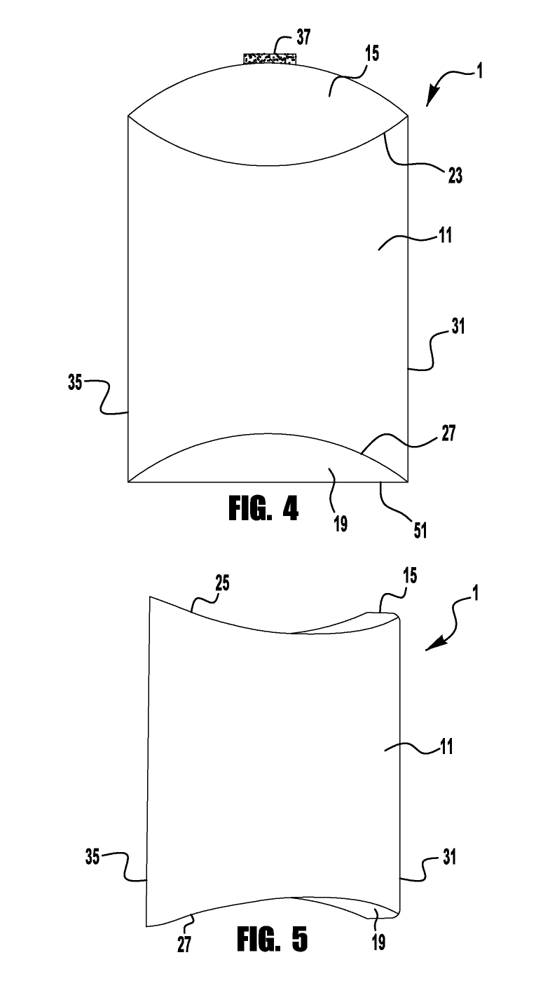

By folding the bottom flaps 19 and 21 at fold lines 51 and 53, the device can be flattened to provide for more compact, efficient portability and storage. The device is shown in this flattened state in FIG. 4.

A used feminine hygiene product can then be disposed into the opening 53 and into the bag 37. To form a seal the top flaps 15 and 17 are then folded downward, as shown in FIG. 5. The sleeve 1 can then be disposed into a trash receptacle or stored until an appropriate receptacle becomes available, without the possibility of fluid or odorous leakage.

Although the invention has been shown and described with respect to a certain preferred embodiment or embodiments, certain equivalent alterations and modifications will occur to others skilled in the art upon the reading and understanding of this specification and the annexed drawings. In particular regard to the various methods and apparatus used in disposing feminine hygiene waste (i.e., that is functionally equivalent), even though not equivalent to the disclosed methods or apparatus which performs the function in the herein illustrated exemplary embodiments of the invention. In addition, while a particular feature of the invention may have been disclosed with respect to only one of several embodiments, such a feature may be combined with one or more features of the other embodiments as may be desired and advantageous for any given or particular application.

1. A disposable feminine hygiene waste disposal device, comprising:
a sleeve having an upper open end and a lower open end;
upper means extending outward from the upper open end for closing the upper open end of the sleeve;
lower means extending outward from the lower open end for closing the lower open end of the sleeve; and
a disposal bag within the sleeve adapted to contain feminine hygiene waste.

2. The disposable feminine hygiene waste disposal device of claim 1 wherein the sleeve has a waterproof interior surface.

3. The disposable feminine hygiene waste disposal device of claim 1 wherein the sleeve has an inner surface containing a deodorizing element.

4. The disposable feminine hygiene waste disposal device of claim 1 wherein the upper means comprises a pair of upper flaps.

5. The disposable feminine hygiene waste disposal device of claim 4 wherein the upper flaps can each fold to engage each other.

6. The disposable feminine hygiene waste disposal device of claim 1 wherein the lower means comprises a pair of lower flaps.

7. The disposable feminine hygiene waste disposal device of claim 6 wherein the lower flaps can each fold to engage each other.

8. The disposable feminine hygiene waste disposal device of claim 7 wherein the lower flaps can be folded when sealed so that the sleeve can be flattened.

9. The disposable feminine hygiene waste disposal device of claim 1 wherein the disposal bag is attached an interior surface of the sleeve by means of an adhesive strip.

10. The disposable feminine hygiene waste disposal device of claim 1 wherein the disposal bag is constructed of waterproof material.

11. The disposable feminine hygiene waste disposal device of claim 1 wherein the disposal bag features a means for sealing the open end of the bag.

12. The disposable feminine hygiene waste disposal device of claim 1 wherein a tab on the disposal bag projects outward from upper open end of the sleeve.

13. A method for disposing of feminine hygiene waste, comprising the steps of:
providing a sleeve having an upper open end with upper flaps extending outward from the upper open end for closing the upper open end of the sleeve and a lower open end with lower flaps extending outward from the lower open end for closing the lower open end of the sleeve;
providing a disposal bag within the sleeve;
closing the lower open end of the sleeve by folding the lower flaps to engage each other;
disposing feminine hygiene waste within the disposable bag;
closing the upper open end of the sleeve by folding the upper flaps to engage each other whereby the disposable bag is contained entirely within the sleeve.

14. The method for disposing of feminine hygiene waste of claim 13 wherein the sleeve has a waterproof interior surface.

15. The method for disposing of feminine hygiene waste of claim 13 wherein the sleeve has an inner surface containing a deodorizing element.

16. The method for disposing of feminine hygiene waste of claim 13 wherein the lower flaps can be folded when sealed so that the sleeve can be flattened.

17. The method for disposing of feminine hygiene waste of claim 13 wherein the disposal bag adheres to an inner surface of the sleeve.

18. The method for disposing of feminine hygiene waste of claim 13 wherein the disposal bag is constructed of waterproof material.

19. The method for disposing of feminine hygiene waste of claim 13 wherein the disposal bag features a means for sealing the open end of the bag.

20. The method for disposing of feminine hygiene waste of claim 13 wherein a tab on the disposal bag projects outward from upper open end of the sleeve.
